(19) United States
(12) Patent Application Publication
(10) Pub. No.: US 2019/0150707 A1
(43) Pub. Date: May 23, 2019

(54) BENDING CONTROL MECHANISM FOR AN ENDOSCOPE

(71) Applicant: Olympus Corporation, Tokyo (JP)

(72) Inventor: Toshihiro Matsui, Lexington, MA (US)

(73) Assignee: Olympus Corporation, Tokyo (JP)

(21) Appl. No.: 16/254,740

(22) Filed: Jan. 23, 2019

Related U.S. Application Data
(63) Continuation of application No. PCT/JP2017/007160, filed on Feb. 24, 2017.

(30) Foreign Application Priority Data
Jul. 25, 2016 (JP) 2016-145723

(51) Int. Cl. A61B 1/005; A61B 1/00

(52) U.S. Cl. CPC A61B 1/0052; A61B 1/00128

(57) ABSTRACT

A bending control mechanism for an endoscope includes a bending control lever controlling a bending portion disposed in an insertion portion of the endoscope. The bending control lever is turnably held on a first proximal end side thereof and is configured to enable the bending portion to bend by tilting the bending control lever on a first distal end side. A first annular surface is formed on the bending control lever. A second annular surface is formed on the bending control lever adjacent to the first annular surface. A third annular surface is formed on the bending control lever adjacent to the second annular surface. An elastic cover has a first attached portion being attached with the first annular surface, a second attached portion being attached with the second annular surface, a third attached portion being attached with the third annular surface, and a cover portion.

### CROSS-REFERENCE TO RELATED APPLICATIONS

This application is a continuation application of PCT Application No. PCT/JP2017/007160 filed on Feb. 24, 2017, which in turn claim priority to the Japanese Patent Application No. 2016-145723 filed Jul. 25, 2016 in Japan which is hereby incorporated by reference in its entirety.

### TECHNICAL FIELD

The disclosed technology generally relates to a bending control mechanism for an endoscope, which performs, by a bending control lever of a bending portion disposed in an insertion portion.

### DESCRIPTION OF THE RELATED ART

To observe a hardly-accessible part inside a subject, such as in the body of a living organism, endoscopes that can be inserted into subjects have already found wide-spread utility, for example, in the medical field. An endoscope of this type has an insertion portion to be inserted into a subject. A bending portion is disposed on a distal end side of the insertion portion, and can be bent freely in desired directions according to remote control to a bending control mechanism disposed in a control portion of the endoscope.

A bending control mechanism of the so-called joystick type that includes a bending control lever tiltable in desired directions is known. In a base portion of such a bending control mechanism of the joystick type, an opening is formed to tilt the bending control lever in desired directions.

In JP 2016-55041A, for example, a rubber boot is disclosed as an elastic cover for sealing such an opening watertight. The rubber boot is in the shape of a cap extending outward in a convex form at a central part thereof, and includes an edge portion of a circular shape in cross-section formed facing an edge portion on the side of the opening. A rod extends as a bending control lever through a hole portion formed through the convex portion of the rubber boot. These rubber boot and rod are connected watertight to each other so that a cleaning solution or the like does not penetrate inside.

However, in addition to being deformed in conjunction with tilting of a bending control lever, an elastic cover of this sort is also deformed under pressure applied to the outside or inside of an endoscope upon conducting autoclave sterilization, a leak test or the like.

It is, therefore, needed for an endoscope to maintain water tightness or the like and ensuring sufficient durability against changes in pressure to be applied to the outside or inside of the endoscope.

With the foregoing circumstances in view, there is need for a bending control mechanism, which can also ensure sufficient durability against changes in pressure to be applied to the endoscope.

### BRIEF SUMMARY OF EMBODIMENTS

A bending control mechanism for an endoscope includes a bending control lever controlling a bending portion disposed in an insertion portion of the endoscope. The bending control lever is turnably held on a first proximal end side thereof and is configured to enable the bending portion to bend by tilting the bending control lever on a first distal end side. A first annular surface is formed on the bending control lever on the first distal end side. A second annular surface is formed on the bending control lever adjacent to a second proximal end side of the first annular surface and has a smaller diameter than the first annular surface. A third annular surface is formed on the bending control lever adjacent to a third proximal end side of the second annular surface and has a greater diameter than the second annular surface. An elastic cover has a first attached portion being attached in close contact with the first annular surface, a second attached portion being attached in close contact with the second annular surface, a third attached portion being attached in close contact with the third annular surface, and a cover portion used to extend over the bending control lever on a fourth proximal end side of the third attached portion enclosing the bending control lever.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1 is a perspective view illustrating the configuration of an endoscope system.

FIG. 2 is a cross-sectional view of a bending control mechanism.

FIG. 3 is a cross-sectional view of an elastic cover insert-molded on a connecting member of a bending control lever.

FIG. 4 is a side view of the connecting member.

FIG. 5 is a side view of the connecting member as viewed from a different direction.

FIG. 6 is a fragmentary cross-sectional view of the bending control mechanism when the bending control lever has been tilt-operated.

FIG. 7 is a fragmentary cross-sectional view of the bending control mechanism in a leak test of an endoscope.

FIG. 8 is a cross-sectional view illustrating a modification of the bending control mechanism.

FIG. 9 is a behavior diagram of the elastic cover in the leak test.

FIG. 10 is a behavior diagram of another elastic cover, and illustrates an example in comparison with FIG. 9.

### DETAILED DESCRIPTION OF THE EMBODIMENTS

In the following description, various embodiments of the technology will be described. For purposes of explanation, specific configurations and details are set forth in order to provide a thorough understanding of the embodiments. However, it will also be apparent to one skilled in the art that the technology disclosed herein may be practiced without the specific details. Furthermore, well-known features may be omitted or simplified in order not to obscure the embodiment being described.

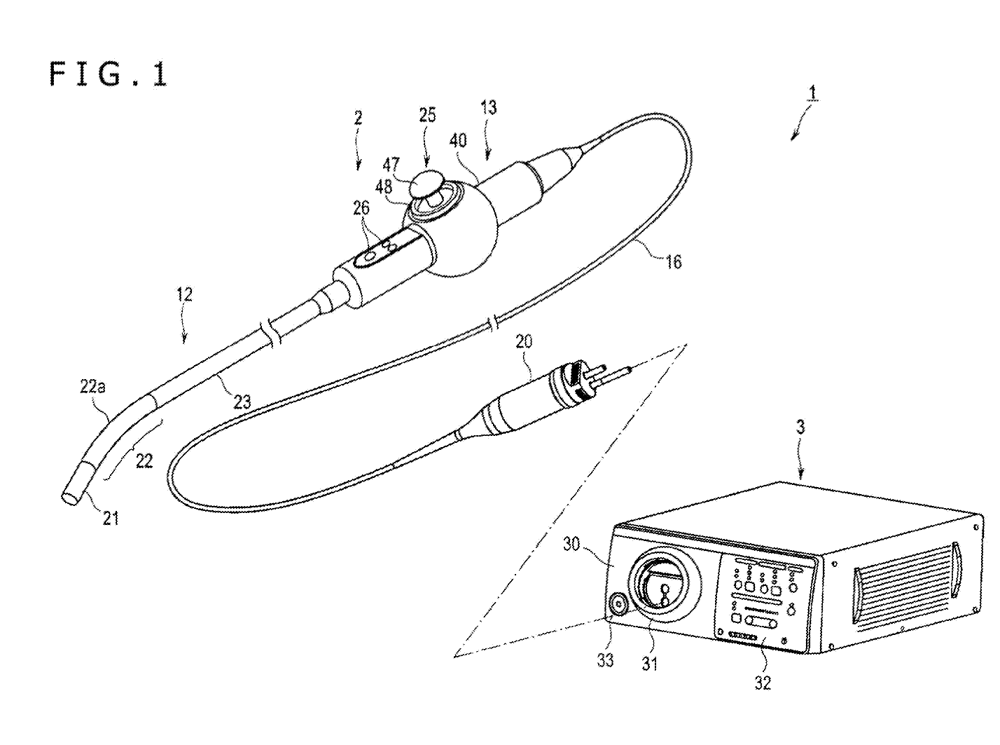

As illustrated in FIG. 1, an endoscope system 1 is configured including, as medical instruments, an endoscope 2 and an image processing device 3 that is used as an external instrument also using as a light source device. The image processing device 3 may also be called “a video processor or camera control unit with a built-in illuminating light source.”

The endoscope 2 is configured primarily including an elongated insertion portion 12, a control portion 13 disposed contiguously on a proximal end of the insertion portion 12, and an endoscope connector 20 to be connected to the image processing device 3.

The control portion 13 and endoscope connector 20 of the endoscope 2 are connected to each other via a flexible cable 16 as a universal cord.

The insertion portion 12 is configured including a tip portion 21 formed from a metal member made of metal, mainly such as stainless steel, a flexibly-bendable bending portion 22, and an elongated rigid tube 23 formed by a metal tube made of stainless steel or the like, all of which are arranged in this order from a distal end side. In other words, the endoscope 2 in this embodiment is a rigid surgical endoscope having the bending portion 22 in the insertion portion 12.

The tip portion 21 includes an unillustrated, built-in imaging portion using a charge-coupled device (CCD) sensor or complementary metal-oxide semiconductor (CMOS) sensor, and a communication cable for drive control and a high-speed transmission optical fiber or the like, which transmits image signals, are arranged extending from this imaging portion.

Inside the bending portion 22, a plurality of unillustrated bending elements is disposed aligned in a row along a longitudinal direction. Through pull or release of unillustrated multiple (for example, four) bending control wires, these bending elements are turned relative to one another, whereby the bending portion 22 can be bent in desired directions including upward, downward, leftward and rightward directions with respect to the imaging portion. In addition, a bendable rubber 22a is also disposed as an outer sheath that covers the bending elements.

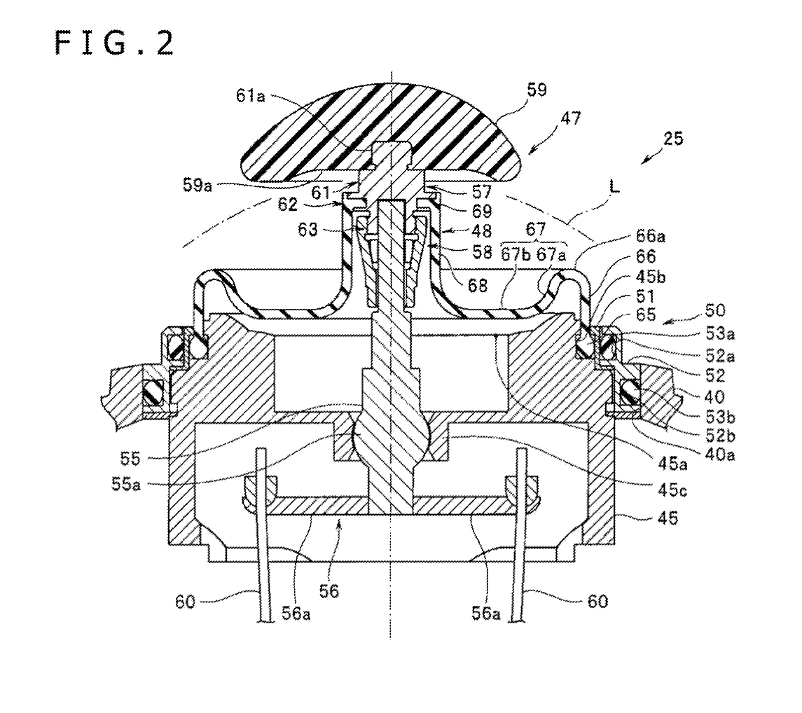

Through the interior of the rigid tube 23, the communication cable and optical transmission fiber, which are arranged extending from the imaging portion in the tip portion 21, a light guide, which transmits illuminating light to the tip portion 21, and the like are inserted by way of the bending portion 22. Individual bending control wires 60 (see FIG. 2), which are connected at distal ends thereof to the most distal bending element in the bending portion 22, are also inserted thorough the interior of the rigid tube 23.

The control portion 13 includes a bending control mechanism 25 for performing remote control of the bending portion 22 via the bending control wires 60, and various switches 26 for controlling the image processing device 3 and the like.

Inserted through the interior of the control portion 13 are built-in components such as the communication cable, optical transmission fiber and light guides, all of which extend from the insertion portion 12 and have been described hereinbefore. These built-in components are connected to the endoscope connector 20 through the interior of the flexible cable 16.

The image processing device 3 includes, on a front wall portion 30 as a control portion for the image processing device 3, a receptacle portion 31 to which the endoscope connector 20 is to be connected, a panel portion 32 for inputting operation and displaying conditions. Inside the image processing device 3, an unillustrated halogen lamp or the like is built as a light source for supplying illuminating light to the endoscope 2.

Next, a description will hereinafter be made in detail about the configuration of the bending control mechanism 25.

As illustrated in FIG. 2, the bending control mechanism 25 of this embodiment is configured including a base portion 45 held in place on a control portion main body 40 of the control portion 13, a bending control lever 47 tiltably supported on the base portion 45, and an elastic cover 48 disposed between the base portion 45 and the bending control lever 47.

The base portion 45 is made of a metal member having, for example, a substantially cylindrical shape, and on a distal end side of the base portion 45, an open end is arranged as an opening 45a through which the bending control lever 47 can be inserted.

The base portion 45 is inserted on a proximal end side in the control portion main body 40 via a base portion insertion hole 40a that is open in the control portion main body 40.

Between an outer periphery on the distal end of the base portion 45 and the base portion insertion hole 40a, a seal portion 50 is interposed to hold the base portion 45 watertight against the control portion main body 40.

Described specifically, the seal portion 50 in this embodiment is configured including a first holder ring 51, which is disposed on an outer peripheral portion on the distal end side of the base portion 45, and a second holder ring 52 disposed between an outer peripheral portion of the first holder ring 51 and an inner peripheral portion of the base portion insertion hole 40a.

Between an inner peripheral wall of the first holder ring 51 and the outer peripheral portion on the distal end side of the base portion 45, the elastic cover 48 is interposed a part thereof as will be described hereinafter, whereby the first holder ring 51 is held in a watertight state against the base portion 45.

A seal groove 52a is formed around in an inner peripheral portion of the second holder ring 52. A seal ring 53a formed of an O-ring or the like is held in the seal groove 52a. This seal ring 53a is pressed against an outer peripheral wall of the first holder ring 51, whereby the second holder ring 52 is connected watertight to the first holder ring 51.

Further, a seal groove 52b is formed around in an outer peripheral portion of the second holder ring 52. A seal ring 53b formed of an O-ring or the like is held in the seal groove 52b. This seal ring 53b is pressed against an inner peripheral wall of the base portion insertion hole 40a, whereby the second holder ring 52 is held watertight against the control portion main body 40.

The bending control lever 47 is configured including a shaft member 55, a wire pulling member 56, a connecting member 57, a tightening member 58, and a finger rest 59. The wire pulling member 56 is disposed on a longitudinal end side, specifically a proximal end side of the shaft member 55. The connecting member 57 is disposed on an opposite longitudinal end side, specifically a distal end side of the shaft member 55. The tightening member 58 attaches the connecting member 57 on the shaft member 55. The finger rest 59 is held on the shaft member 55 via the connecting member 57.

The shaft member 55 is disposed inside the base portion 45 on the longitudinal end side, specifically the proximal end side, and extends to an outside, in other words, beyond a distal end of the base portion 45 through the opening 45a.

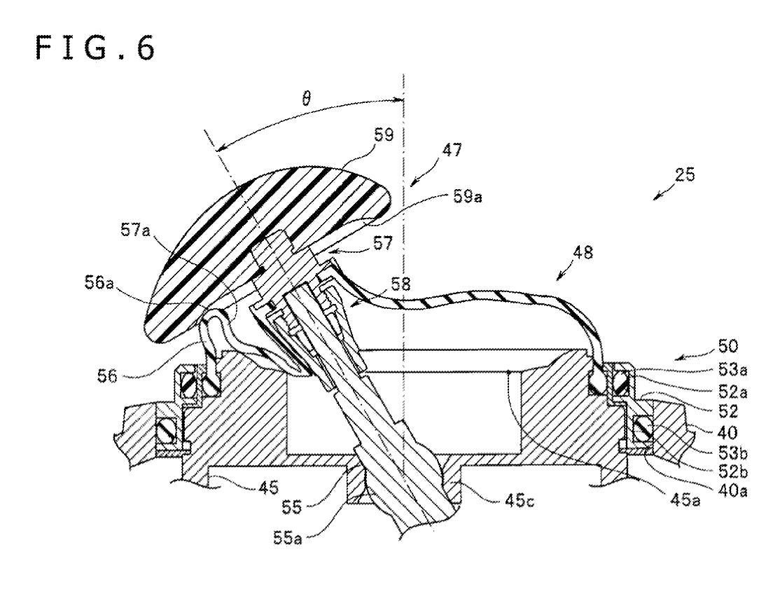

On an intermediate portion of the shaft member 55, for example, between the wire pulling member 56 and the connecting member 57, a spherical body 55a is disposed. This spherical body 55a is slidably connected to a spherical body bearing 45c disposed inside the base portion 45. As a consequence, the shaft member 55 is turnably held with the spherical body 55a acting as a fulcrum, and can be tilted in desired directions from a neutral state (see FIG. 2), in which the shaft member 55 extends upright along an axis of the base portion 45, to a predetermined angle, specifically a maximum tilt angle θ (see FIG. 6).

The wire pulling member 56 is made of, for example, a plate-shaped member having four arm portions 56a extending in different directions from one another. In this embodiment, more specifically, the wire pulling member 56 is made of a cruciform plate-shaped member with the angles between the mutually-adjacent arm portions 56a being set at 90 degrees. The wire pulling member 56 is attached to a central part on the proximal end side of the shaft member 55. As a consequence, each arm 56a is displaceable on a distal end side in conjunction with tilting motion of the shaft member 55. It is to be noted that in FIG. 2, only two of the four arm portions 56a are illustrated.

To the distal end sides of the respective arms portions 56a, the respective control wires 60 which extend from the side of the insertion portion 12 are connected at proximal ends. As a consequence, the wire pulling member 56 can pull or release the respective bending control wires 60 in conjunction with tilt operation to the shaft member 55, in other words, the bending control lever 47 so that the bending portion 22 can be bent in a desired direction. Therefore, the wire pulling member 56 is configured, for example, to enable bending of the bending portion 22 in an upward or downward direction when the shaft member 55, in other words, the bending control lever 47 is tilted in a forward or rearward direction, and to enable bending of the bending portion 22 in a leftward or rightward direction when the shaft member 55, in other words, the bending control lever 47 is tilted in a leftward or rightward direction.

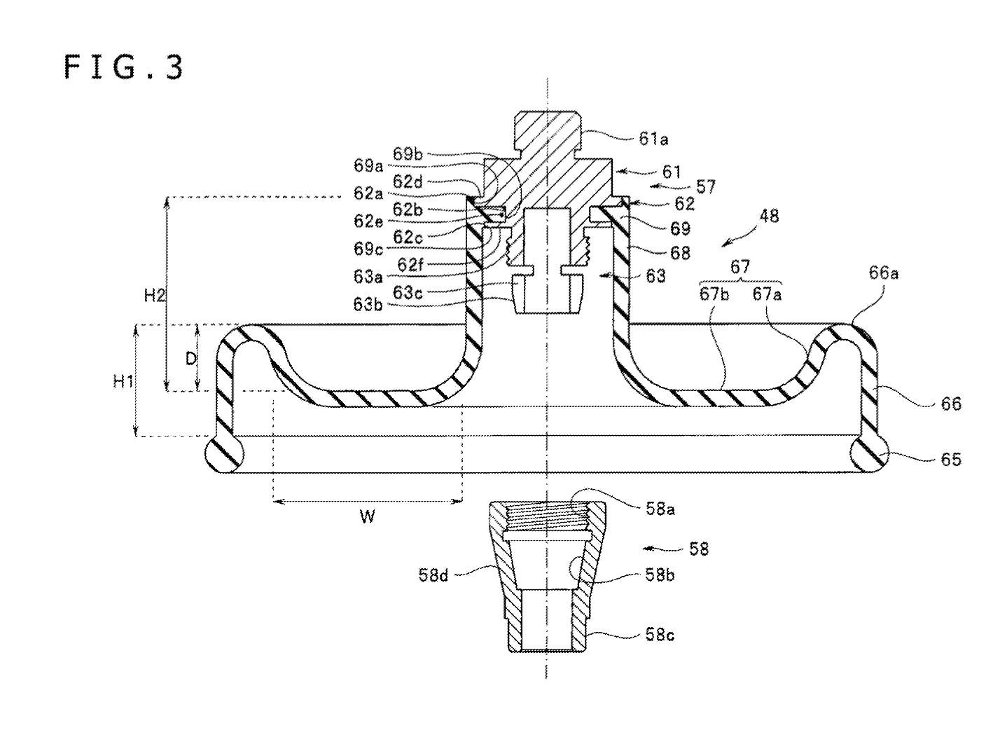

As illustrated in FIGS. 3 to 5, for example, the connecting member 57 is made of an integrally-formed metal member, in which a head portion 61 with the finger rest 59 attached thereon, an insert portion 62 embedded in a part of the elastic cover 48, and a cylindrical portion 63 with the shaft member 55 inserted on the distal end side are contiguously arranged in this order from a distal end side.

The head portion 61 has a substantially circular columnar shape, and includes at a top portion thereof a protruding portion 61a for attaching the finger rest 59.

On the insert portion 62, a first annular surface 62a of a diameter greater than that of the head portion 61, a second annular surface 62b of a diameter different from that of the first annular surface 62a, and a third annular surface 62c of a diameter different from that of the second annular surface 62b are formed adjacent one another in this order from the distal end side.

Described specifically, an outward flange 62d of a diameter greater than that of the head portion 61 is disposed around on the distal end side of the insert portion 62. An outer peripheral wall of the outward flange 62d is arranged as the first annular surface 62a. Further, a recessed groove 62e of a diameter smaller than that of the outward flange 62d is formed (i) around in the insert portion 62, (ii) adjacent the outward flange 62d, and (iii) on a side closer to a proximal end of the insert portion 62 than the outward flange 62d. The recessed groove 62e has a bottom wall arranged as the second annular surface 62b. Furthermore, an outward flange 62f of a diameter greater than that of the recessed groove 62e is disposed (i) around on the insert portion 62, (ii) adjacent the recessed groove 62e, and (iii) on a side closer to the proximal end of the insert portion 62 than the recessed groove 62e. The outward flange 62f has an outer peripheral wall arranged as the third annular surface 62c.

Here, to provide enhanced interfacial adhesion upon insert-molding the elastic cover 48, the first to third annular surfaces 62a to 62c are desired to have a surface roughness set at least equal to that of the remaining parts, specifically equal to or higher than that of the remaining parts of the connecting member 57, in other words, the bending control lever 47.

The outer diameters of the first to third annular surfaces 62a to 62c are needed to be different between each two mutually-adjacent ones of them. Therefore, it is possible to form, for example, the second annular surface 62b by an outer peripheral wall of an outward flange of a diameter greater than those of the respective flanges 62d, 62f that form the first and third annular surfaces 62a, 62c, respectively. In addition, the third annular surface 62c may be omitted as desired.

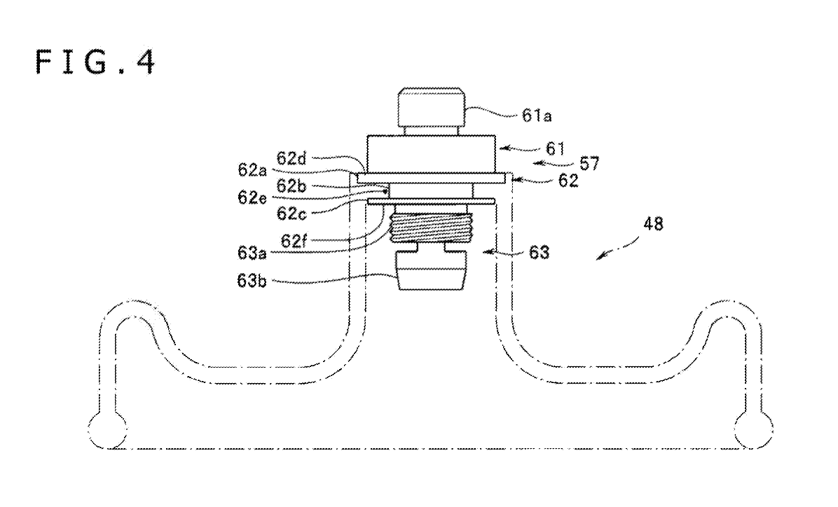

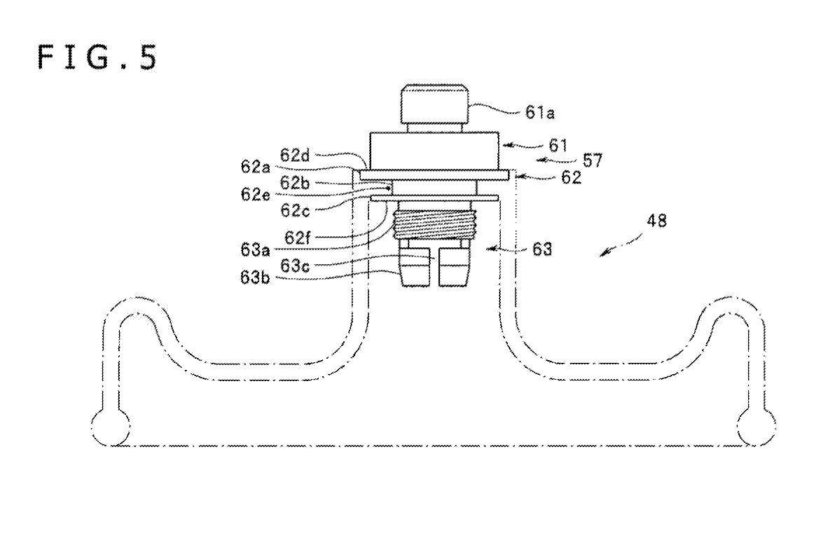

As illustrated in FIGS. 2 and 3, the cylindrical portion 63 makes up a so-called collet chuck holder together with the tightening member 58. Accordingly, an externally threaded portion 63a is formed on an outer periphery of a distal end side of the cylindrical portion 63. Further, on the outer periphery of a proximal end side of the cylindrical portion 63, a tapered surface 63b is formed with a diameter increasing from the proximal end side toward the distal end side. Furthermore, on the proximal end side of the cylindrical portion 63, slits 63c are formed to allow deformation of the cylindrical portion 63 in radial directions on the proximal end side (see FIGS. 4 and 5).

The tightening member 58 is made of a cylindrical metal member to be externally attached to the cylindrical member 63 of the connecting member 57. On an inner periphery of a distal end side of the tightening member 58, an internally threaded portion 58a which is engageable with the externally threaded portion 63a of the cylindrical portion 63 is formed.

On an inner periphery of the tightening member 58 on a side closer to the proximal end than the internally threaded portion 58a, a tapered surface 58b is formed with a diameter increasing from the proximal end side toward the distal end side. When the internally threaded portion 58a is engaged with the externally threaded portion 63a of the cylindrical portion 63, the internally threaded portion 58a is brought into contact with the tapered surface 63b of the cylindrical portion 63. The cylindrical portion 63 is deformed in radially inward directions as the threaded engagement of the internally threaded portion 58a with the externally threaded portion 63a proceeds. As a consequence, the tightening member 58 can firmly attach the connecting member 57 on the shaft member 55. Here, serration machining may be applied to a distal end side of the shaft member 55 and also to the cylindrical portion 63 to enhance the attaching of the connecting member 57 in turning directions about its axis.

Disposed on an outer periphery of a distal end portion of the tightening member 58 is a nut portion 58c, with which an unillustrated tool is brought into engagement upon threaded engagement with the externally threaded portion 63a. In addition, on a side closer to the distal end than the nut portion 58c, a tapered surface 58d is formed on an outer periphery of the tightening member 58 with a diameter increasing from the proximal end side toward the distal end side.

As a result of fastening of the tightening member 58 of such a configuration on the connecting member 57, the bending control lever 47 has the tapered surface on a side closer to the proximal end side than the insert portion 62. Specifically, the bending control lever 47 has the tapered surface 58d of an outer diameter, which becomes smaller from the distal end side toward the proximal end side. The tapered surface 58d is formed in a region enclosed by the elastic cover 48 as will be described hereinafter.

Here, the nut portion 58c and tapered surface 58d disposed on the outer peripheral side of the tightening member 58 may desirably be machined, at respective edge portions thereof, in a rounded shape by chamfering or the like.

The finger rest 59 is made of an umbrella-shaped member with which operator's fingers can be kept in contact, and is attached on the shaft member 55 via the protruding portion 61a disposed on the head portion 61 of the fastening member 57. The finger rest 59 is formed with an outer diameter greater than that of the shaft member 55, and on a back side of the finger rest 59, a concave portion 59a is formed to avoid interference with the elastic cover 48.

The elastic cover 48 is made of a flexible rubber molding disposed to close the opening 45a of the base portion 45. This elastic cover 48 is made of an integral molding including a watertight attached portion 65, an outer peripheral wall portion 66, a valley portion 67, an inner peripheral wall portion 68 and a central portion 69, all of which are arranged in this order from an outer peripheral side of the elastic cover 48. The watertight attached portion 65 is formed on and along the peripheral edge portion of the elastic cover 48, and is attached watertight on the base portion 45. The outer peripheral wall portion 66 rises from the watertight attached portion 65 to a top portion 66a set at a first height H1. The valley portion 67 is disposed contiguously on an inner peripheral side of the outer peripheral wall portion 66. The inner peripheral wall portion 68 is disposed contiguously on an inner peripheral side of the valley portion 67, and rises to a second height H2 higher than the first height H1. The central portion 69 is disposed contiguously on an inner peripheral side of the inner peripheral wall portion 68, and is attached on the bending control lever 47. Of these individual elements of the elastic cover 48, the series of portions from the outer peripheral wall portion 66 to the inner peripheral wall portion 68 make up the cover portion that encloses the bending control lever 47.

The watertight attached portion 65 is made, for example, of an O-ring shaped member formed on an outermost periphery of the elastic cover 48. This watertight attached portion 65 is held in a seal groove 45b formed around in an outer peripheral portion of a distal end side of the base member 45. The watertight attached portion 65 held in the seal groove 45b is pressed against the inner peripheral wall of the first holder ring 51, whereby the base portion 45 is connected in a watertight state to the control portion main body 40 and the elastic cover 48 is inseparably held on the base member 45, in other words, the control portion main body 40.

The outer peripheral wall portion 66 is made of a substantially cylindrical member integrally molded so that the outer peripheral wall portion 66 rises from the watertight attached portion 65 toward a distal end of an axis of the bending control lever 47.

The valley portion 67 is made of a substantially toroidal member integrally molded so that a depression is formed from the top portion 66a toward the opening 45a.

This valley portion 67 is configured including a descending portion 67a, which extends in a descending direction from the top portion 66a of the outer peripheral wall portion 66, and a bottom portion 67b, which forms a planar portion on an inner peripheral side of the descending portion 67a.

Here, as indicated in FIG. 3, for example, the descending portion 67a has a depth set at a predetermined depth D smaller than the first height H1. Further, the bottom portion 67b has a radial width set, for example, at a predetermined uniform width W over an entire periphery thereof, in other words, over front-to-rear and left-to-right directions of the control portion 13.

The inner peripheral wall portion 68 is made of a substantially cylindrical member integrally molded so that the inner peripheral wall portion 68 rises from an inner periphery of the valley portion 67 toward the distal end of the axis of the bending control lever 47.

In the central portion 69, the insert portion 62 of the connecting member 57 is embedded by insert molding on an inner peripheral side of the central portion 69. As a consequence, the bending control lever 47 is connected watertight with the elastic cover 48.

Specifically, as illustrated in FIG. 3, for example, the central portion 69 is configured including a first attached portion 69a attached in close contact with the first annular surface 62a, a second attached portion 69b attached in close contact with the second annular surface 62b, and a third attached portion 69c attached in close contact with the third annular surface 62c, the first annular surface 62a, second annular surface 62b and third annular surface 62c being all formed on the insert portion 62.

The first to third attached portions 69a to 69c are formed copying the shape of the insert portion 62 upon insert molding. Owing to the attaching of the first to third attached portions 69a to 69c in close contact with the first to third annular surfaces 62a to 62c, respectively, the elastic cover 48 is firmly connected with high water tightness to the bending control lever 47.

Here, between terminating ends, specifically the distal end of the insert portion 62 and a distal end of the central portion 69, the first attached portion 69a is attached in close contact with the first annular surface 62a so that their bonded interface extends in the direction of the axis of the bending control lever 47. Further, on a proximal end side of a bonded portion between the first annular surface 62a and the first attached portion 69a, a bonded portion between the second annular surface 62b, which has the diameter different from that of the first annular surface 62a, and the second attached portion 69b and a bonded portion between the third annular surface 62c, which has the diameter different from that of the second annular surface 62b, and the third attached portion 69c are disposed contiguously with one another, so that the bonded interface between the insert portion 62 and the central portion 69 has a labyrinth structure in cross-section. If the third annular surface 62c of the insert portion 62 is omitted, the third attached portion 69c of the central portion 69 is also omitted.

Here, in the elastic cover 48 as described hereinbefore, the first height H1 to the top portion 66a of the outer peripheral wall portion 66 is set so that the top portion 66a in a neutral state is located on a side inner than a locus L of the finger rest 59 during tilting of the bending control lever 47 (see FIG. 2).

The first height H1 to the top portion 66a of the outer peripheral wall 66 is set at a height so that the finger contact 59 does not come into contact with the top portion 66a even if the elastic cover 48 is deformed in conjunction with tilting of the shaft member 55 to a predetermined angle, specifically a maximum tilt angle θ by the bending control lever 47 (see FIG. 6).

The first height H1 to the top portion 66a of the outer peripheral wall 66 is set at a height so that the outer peripheral wall portion 66 and the descending portion 67a do not come into contact with each other even if the elastic cover 48 is deformed in conjunction with tilting of the shaft member 55 to the predetermined angle θ via the bending control lever 47 (see FIG. 6).

The elastic cover 48 in this embodiment is set so that the total length, in other words, the distance from the peripheral edge portion, specifically the watertight attached portion 65 to the central portion 69 when the outer peripheral wall portion 66, valley portion 67 and inner peripheral wall portion 68 have been spread unfolded becomes greater than the linear distance between the peripheral edge portion, specifically the watertight attached portion 65 and the central portion 69 when the shaft member 55 has been tilted to the predetermined angle, specifically the maximum tilt angle θ (see FIG. 6).

The setting of the first height H1 as described hereinbefore can be conducted through experiments, simulations and the like based on specifications such as, for example, the depth D of the descending portion 67a, the width W of the bottom portion 67b, and the elasticity and shape of a resin material that makes up the elastic cove 48. In other words, especially, the first height H1, the depth D of the descending portion 67a and the width W of the bottom portion 67b are set in a mutually-correlated manner so that the respective requirements described hereinbefore are met upon tilting of the bending control lever 47.

According to the embodiment as described hereinbefore, bending operation to the bending control lever 47 can be performed with good operability by (I) configuring the elastic cover 48 with (i) the watertight attached portion 65 as the peripheral edge portion to be attached watertight on the base portion 45, (ii) the outer peripheral wall portion 66 rising from the watertight attached portion 65 to the top portion 66a formed at the first height H1 where the finger rest 59 does not come into contact with the elastic cover 48 when the shaft member 55 has been tilted to the predetermined angle θ, the valley portion 67 disposed contiguously on an inner peripheral side of the outer peripheral wall portion 66, (iii) the inner peripheral wall portion 68 disposed contiguously on an inner peripheral side of the valley portion 67 and rising to the second height H2 higher than the first height H1, and (iv) the central portion 69 disposed contiguously on an inner peripheral side of the inner peripheral wall portion 68 and attached on the bending control lever 47, and (II) closing watertight the opening 45a of the base portion 45 with the elastic cover 48 as described hereinbefore.

In other words, it is possible to avoid interference between the elastic cover 48 and the finger rest 59 upon tilting the bending control lever 47 by setting the height from the watertight attached portion 65 as the peripheral edge portion to the top portion 66a of the outer peripheral wall portion 66 at the first height H1. The outer peripheral wall portion 66 and inner peripheral wall portion 68 are disposed on an outer peripheral side and inner peripheral side, respectively, via the valley portion 67 in the elastic cover 48. The finger rest 59 does come into contact with the top portion 66a when the shaft member 55 has been tilted to the predetermined maximum tilt angle θ. A reaction force, which the bending control lever 47 receives from the elastic cover 48 via the finger rest 59 upon tilting the bending control lever 47, can hence be reduced so that bending operation to the bending control lever 47 can be performed with good operability.

The height of the inner peripheral wall portion 68 is hardly expected to interfere with the finger rest 59 or the like. The height of the inner peripheral wall portion 68 is set at the second height H2 higher than the first height H1 so as to ensure a sufficient elastically-deformable region for the inner peripheral wall portion 68. Therefore, it is also possible to effectively distribute a load, which the inner peripheral wall portion 68 receives directly from the bending control lever 47 upon tilting. Resistance to tilting operation to the bending control lever 47 can hence be reduced further.

Here, the first height H1 is set under an additional condition that the inner peripheral wall portion 68 and the descending portion 67a are not brought into contact with each other even if the elastic cover 48 is deformed in conjunction with tilting of the shaft member 55 to the predetermined angle θ via the bending control lever 47. In this case, it is possible to prevent an increase in reaction force through deformation of the elastic cover 48 although such an increase would otherwise occur by interference between the inner peripheral wall portion 68 and the descending portion 67a upon tilting the bending control lever 47. It is, therefore, possible to reduce a reaction force, which is to be received from the elastic cover 48 via the shaft member 55 upon tilting the bending control lever 47, and to perform bending operation to the bending control lever 47 with still better operability.

The total length TL is defined as the distance from the peripheral edge portion, specifically the watertight attached portion 65 to the central portion 69 upon unfolded spreading of the outer peripheral wall portion 66, valley portion 67 and inner peripheral wall portion 68. The linear distance LD is defined as the distance between the peripheral edge portion, specifically the watertight attached portion 65 and the central portion 69 upon tilting of the shaft member 55 to the predetermined angle, specifically the maximum tilt angle θ. In this case, the total length TL is set greater than the linear distance LD. Therefore, it is possible to reduce a tension to be produced through deformation of the elastic cover 48, and hence to realize bending operation to the bending control lever 47 with good operability.

According to the embodiment as described hereinbefore, sufficient durability can also be ensured against changes in pressure to be applied to the endoscope 2 by connecting the elastic cover 48 watertight to the bending control lever 47. The bending control lever 47 includes the first annular surface 62a and the second annular surface 62b. The first annular surface 62a is formed at the predetermined position on the distal end side of the bending control lever 47. The second annular surface 62b is formed adjacent the proximal end side of the first annular surface 62a and has the diameter different from the first annular surface 62a. The elastic cover 48 includes the first attached portion 69a, the second attached portion 69b, and the cover portion. The first attached portion 69a is to be attached in close contact with the first annular surface 62a. The second attached portion 69b is to be attached in close contact with the second annular surface 62b. The cover portion, specifically the outer peripheral wall portion 66, valley portion 67 and inner peripheral wall portion 68 is disposed extending on a side closer to the proximal end of the bending control lever 47 than the second attached portion 69b and enclosing the bending control lever 47.

By attaching the first attached portion 69a of the elastic cover 48 in close contact with the first annular surface 62a formed on the bending control lever 47 at a terminating end, specifically a distal end of the bonded portion between the bending control lever 47 and the elastic cover 48, their bonded interface can be arranged along the direction of the axis of the bending control lever 47. In addition, by forming the bonded portion between the second annular surface 62b, which is different in diameter from the first annular surface 62a, and the second attached portion 69b contiguously on a proximal end side of the bonded portion between the first annular surface 62a and the first attached portion 69a, the cross-sectional shape having the labyrinth structure can be formed at the bonded interface between the bonding control lever 47 and the elastic cover 48. As a consequence, even if the cover portion, which extends on the side closer to the proximal end of the bending control lever 47 than the bonded portion between the insert portion 62 and the central portion 69, undergoes substantial elastic deformation under an external pressure, an internal pressure or the like applied to the endoscope 2, a stress by the elastic deformation can be prevented from being transmitted to the terminating end of the bonded interface. It is, therefore, possible to prevent occurrence of separation or the like at the bonded interface between the bending control lever 47 and the elastic cover 48 and also to ensure sufficient durability against changes in pressure to be applied to the endoscope 2.

Here, the durability can be more effectively enhanced by further forming the bonded portion between the third annular surface 62c, which is different in diameter from the second annular surface 62b, and the third attached portion 69c contiguously on a proximal end side of the bonded portion between the second annular surface 62b and the second attached portion 69b.

Owing to the inclusion of the tapered surface 58d, the outer diameter of which decreases toward the proximal end of the bending control lever 47, in the region where the bending control lever 47 is enclosed by the cover portion of the elastic cover 48, it is possible to prevent formation of a stepped portion or the like, which causes a sudden change in outer diameter, on the bending control lever 47 on a side closer to the proximal end than the bonded portion between the insert portion 62 and the elastic cover 48, in other words, the insert portion 62. Even if the elastic cover 48 is pressed against the bending control lever 47 under an external pressure or the like applied to the endoscope 2, for example, during autoclave sterilization or the like, damage or the like to the elastic cover 48 can be prevented although such damage or the like would otherwise occur through contact with a stepped portion or the like.

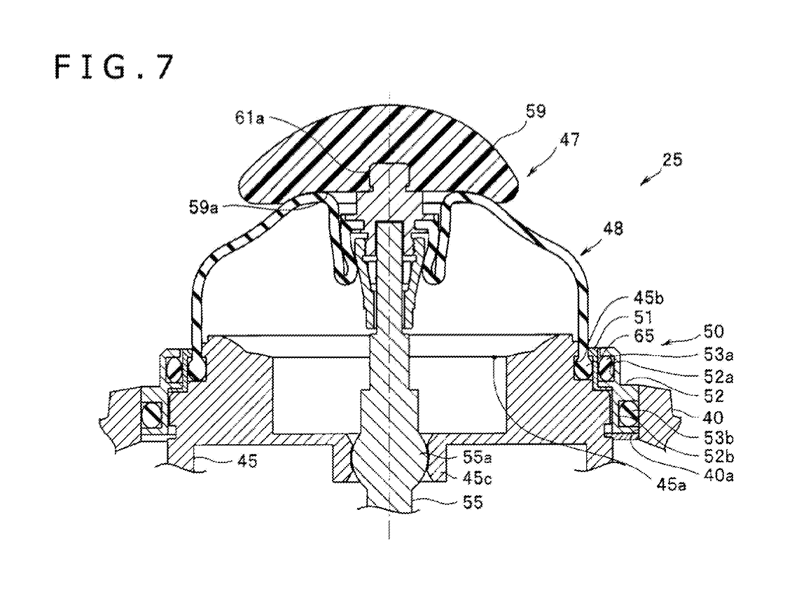

As illustrated in FIG. 7, for example, the elastic cover 48 expands under an internal pressure or the like applied to the endoscope 2 when a leak test or the like is conducted on the endoscope 2. Owing to the interposition of the valley portion 67, which includes the planar bottom portion 67b, between the outer peripheral wall portion 66 and the inner peripheral wall portion 68, the elastic cover 48 in this embodiment can avoid abrupt deformation under the internal pressure or the like so applied, and therefore can exhibit enhanced durability.

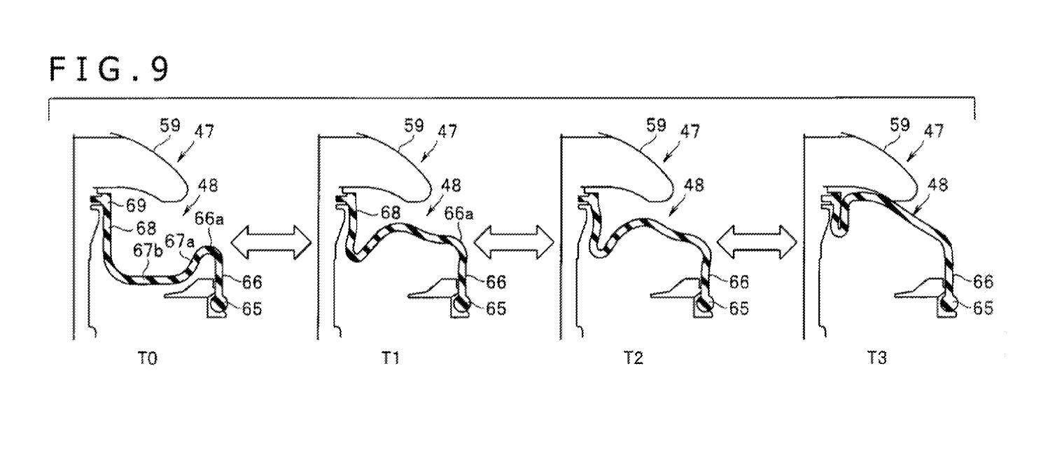

Described specifically, as illustrated in FIG. 9, for example, the elastic cover 48 having the planar bottom portion 67b begins to deform from a relatively low pressure level upon reception of an internal pressure at the bottom portion 67b, so that the deformation gradually proceeds as the pressure rises (see timings T0 to T3). If the internal pressure is released, on the other hand, the elastic cover 48 gradually returns toward its original shape from the deformed shape as the pressure decreases. As a consequence, the elastic cover 48 definitely restores its initial shape upon completion of a leak test.

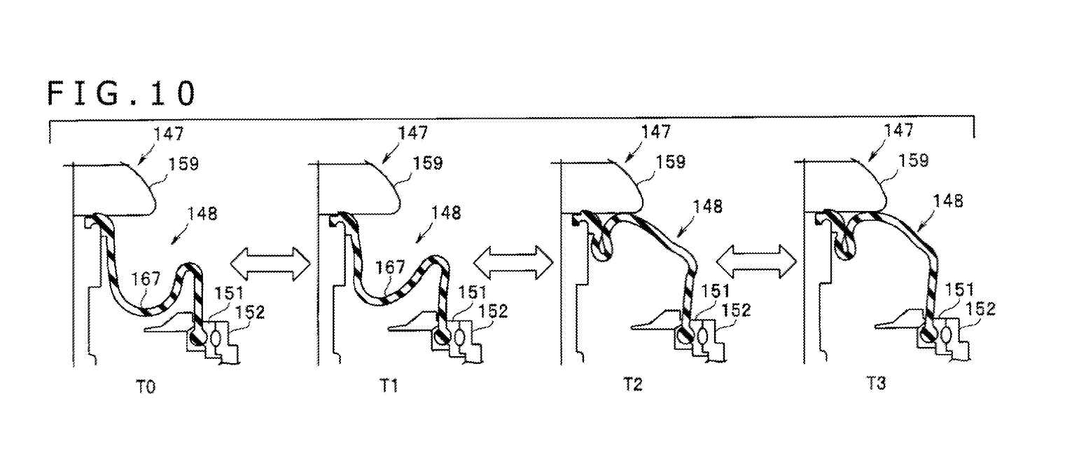

On the other hand, as illustrated in a comparative example of FIG. 10, for example, an elastic cover 148 with a valley portion 167 formed as a whole by a concave curved portion is suppressed from deformation under a relatively low level of internal pressure (see timings T0 and T1), but at the moment of an increase in pressure beyond a certain level, the shape of the valley portion 167 is abruptly reversed (see timings T2 and T3). If the internal pressure is released, on the other hand, the elastic cover 148 remains deformed despite a decrease in pressure. As a consequence, upon completion of a leak test, the elastic cover 148 fails to restore its initial shape, and remains expanded. If a bending control lever 147 is operated in this state, the elastic cover 148 is caught between a finger rest 159 and first and second holder rings 151, 152. Then, the reaction force that the bending control lever 147 receives from the elastic cover 148 via the finger rest 159 increases, and moreover a potential problem arises in that the elastic cover 148 may be damaged.

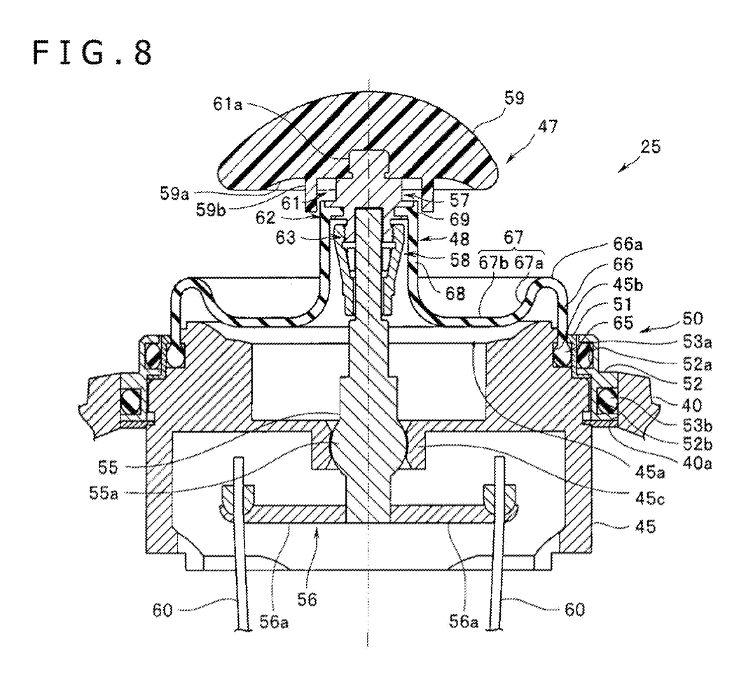

For allowing the elastic cover 48 to promptly restore its original shape upon release of an internal pressure applied to the endoscope 2 in a leak test or the like, it is possible, as illustrated in FIG. 8, for example, to dispose a short cylindrical wall portion 59b, which extends from a proximal end side of the finger rest 59, in proximity to the inner peripheral wall portion 68.

Now, the bending control mechanism 25 described hereinbefore includes, for example, the base portion 45 formed in a cylindrical shape, and as viewed in plan, is formed in a circular shape substantially symmetrical over the entire periphery thereof. For example, in view of the tendency that the operability in leftward and rightward directions is generally lower than that in forward and rearward directions when operating the bending control lever 47 while holding the control portion 13, the bending control mechanism 25 can be formed in a shape asymmetrical between forward and rearward directions and leftward and rightward directions as viewed in plan. Described specifically, especially to improve the operability of tilting operation in leftward and rightward directions to the bending control lever 47, the bending control mechanism 25 can be formed, for example, in a substantially oval shape having a major axis in forward and rearward directions of the control portion 13 and a minor axis in leftward and rightward directions of the control portion 13 as viewed in plan.

The present disclosure should not be limited to the respective embodiments descried hereinbefore, and various modifications and alterations are feasible. Such modifications and alterations should also fall within the technical scope of the present disclosure.

For example, the description is made regarding one example of an application of the present disclosure to the rigid endoscope including the rigid tube 23 in the insertion portion 12 in the embodiments described hereinbefore. The present disclosure should not be limited to such an example, and obviously can be also applied to a flexible endoscope including a flexible tube in the insertion portion 12.

In sum, one aspect of the disclosed technology is directed to a bending control mechanism for an endoscope that comprises a bending control lever controlling a bending portion disposed in an insertion portion of the endoscope. The bending control lever is turnably held on a first proximal end side thereof and is configured to enable the bending portion to bend by tilting the bending control lever on a first distal end side. A first annular surface is formed on the bending control lever on the first distal end side. A second annular surface is formed on the bending control lever adjacent to a second proximal end side of the first annular surface and having a smaller diameter than the first annular surface. A third annular surface is formed on the bending control lever adjacent to a third proximal end side of the second annular surface and having a greater diameter than the second annular surface. An elastic cover having a first attached portion is attached in close contact with the first annular surface. A second attached portion is attached in close contact with the second annular surface. A third attached portion is attached in close contact with the third annular surface. A cover portion is used to extend over the bending control lever on a fourth proximal end side of the third attached portion enclosing the bending control lever.

Another aspect of the disclosed technology is directed to a bending control mechanism for an endoscope comprises a bending control lever controlling a bending portion disposed in an insertion portion of the endoscope. The bending control lever is turnably held on a first proximal end side thereof and is configured to enable bending the bending portion by tilting the bending control lever on a first distal end side. A first annular surface is formed on the bending control lever on the first distal end side. A second annular surface is formed on the bending control lever adjacent to a second proximal end side of the first annular surface and having a greater diameter than the first annular surface. A third annular surface is formed on the bending control lever adjacent to a third proximal end side of the second annular surface and having a smaller diameter than the second annular surface. An elastic cover having a first attached portion is attached in close contact with the first annular surface. A second attached portion is attached in close contact with the second annular surface. A third attached portion is attached in close contact with the third annular surface. A cover portion is used to extend over the bending control lever on a fourth proximal end side of the third attached portion enclosing the bending control lever.

A further aspect of the disclosed technology is directed to an endoscope comprises an insertion portion having respective first proximal and distal ends. The insertion portion includes a bendable portion. A control portion is attached on the proximal end of the insertion portion. The control portion includes a base. A bending control lever is tiltably supported on the base. The bending control lever includes a shaft, a finger rest, and a connecting member that disposed between the shaft and the finger rest. The connecting member includes a first annular surface having a second proximal end side. A second annular surface is formed on the second proximal end side of the first annular surface and having a different diameter of the first annular surface. A third annular surface is formed on a third proximal end side of the second annular surface and having a different diameter of the second annular surface. An elastic cover is disposed between the base and the bending control lever. The elastic cover includes a first attached portion attached to the first annular surface. A second attached portion is attached to the second annular surface. A third attached portion is attached to the third annular surface. A cover portion is used to extend over the bending control lever on a fourth proximal end side of the third attached portion enclosing the bending control lever.

While various embodiments of the disclosed technology have been described above, it should be understood that they have been presented by way of example only, and not of limitation. Likewise, the various diagrams may depict an example schematic or other configuration for the disclosed technology, which is done to aid in understanding the features and functionality that can be included in the disclosed technology. The disclosed technology is not restricted to the illustrated example schematic or configurations, but the desired features can be implemented using a variety of alternative illustrations and configurations. Indeed, it will be apparent to one of skill in the art how alternative functional, logical or physical locations and configurations can be implemented to implement the desired features of the technology disclosed herein.

Although the disclosed technology is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one”, “one or more” or the like; and adjectives such as “conventional”, “traditional”, “normal”, “standard”, “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more”, “at least”, “but not limited to” or other like phrases in some instances shall not be read to mean that the narrower case is intended or required in instances where such broadening phrases may be absent. Additionally, the various embodiments set forth herein are described in terms of exemplary schematics, block diagrams, and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular configuration.

What is claimed is:

1. A bending control mechanism for an endoscope comprising:
a bending control lever controlling a bending portion disposed in an insertion portion of the endoscope, the bending control lever being turnably held on a first proximal end side thereof and being configured to enable the bending portion to bend by tilting the bending control lever on a first distal end side;
a first annular surface being formed on the bending control lever on the first distal end side;
a second annular surface being formed on the bending control lever adjacent to a second proximal end side of the first annular surface and having a smaller diameter than the first annular surface;
a third annular surface being formed on the bending control lever adjacent to a third proximal end side of the second annular surface and having a greater diameter than the second annular surface; and
an elastic cover having a first attached portion being attached in close contact with the first annular surface, a second attached portion being attached in close contact with the second annular surface, a third attached portion being attached in close contact with the third annular surface, and a cover portion used to extend over the bending control lever on a fourth proximal end side of the third attached portion enclosing the bending control lever.

2. The bending control mechanism of claim 1, wherein
the cover portion encloses a tapered surface having an outer diameter that decreases toward the first proximal end side of the bending control lever.

3. The bending control mechanism of claim 1, wherein the first annular surface, second annular surface and third annular surface each of which has a surface roughness set equal to or higher than remaining parts of the bending control lever.

4. The bending control mechanism of claim 1, wherein the elastic cover has
a watertight attached portion that is attached on a side of a control portion of the endoscope along a peripheral edge, and
a planar portion disposed between the third attached portion and the watertight attached portion in a bottom of the elastic cover, the planar portion forming a depression in a proximity to a side of the control portion of the endoscope.

5. The bending control mechanism of claim 1, further comprising:
a finger rest used by an operator to manipulate the bending portion, the finger rest being disposed on the first distal end side of the bending control lever; and
a convex portion formed on the finger rest and extending toward the first proximal end side of the bending control lever.

6. A bending control mechanism for an endoscope comprising:
a bending control lever controlling a bending portion disposed in an insertion portion of the endoscope, the bending control lever being turnably held on a first proximal end side thereof and being configured to enable bending the bending portion by tilting the bending control lever on a first distal end side;
a first annular surface being formed on the bending control lever on the first distal end side;
a second annular surface being formed on the bending control lever adjacent to a second proximal end side of the first annular surface and having a greater diameter than the first annular surface;
a third annular surface being formed on the bending control lever adjacent to a third proximal end side of the second annular surface and having a smaller diameter than the second annular surface; and
an elastic cover having a first attached portion being attached in close contact with the first annular surface, a second attached portion being attached in close contact with the second annular surface, a third attached portion being attached in close contact with the third annular surface, and a cover portion used to extend over the bending control lever on a fourth proximal end side of the third attached portion enclosing the bending control lever.

7. An endoscope comprising:
an insertion portion having respective first proximal and distal ends, the insertion portion including a bendable portion; and
a control portion attached on the proximal end of the insertion portion;
the control portion including:
a base;
a bending control lever being tiltably supported on the base, the bending control lever including a shaft, a finger rest, and a connecting member that disposed between the shaft and the finger rest, the connecting member having:
a first annular surface having a second proximal end side;
a second annular surface formed on the second proximal end side of the first annular surface and having a different diameter of the first annular surface;
a third annular surface formed on a third proximal end side of the second annular surface and having a different diameter of the second annular surface; and
an elastic cover being disposed between the base and the bending control lever, the elastic cover including:
a first attached portion attached to the first annular surface;
a second attached portion attached to the second annular surface;
a third attached portion attached to the third annular surface; and
a cover portion used to extend over the bending control lever on a fourth proximal end side of the third attached portion enclosing the bending control lever.

8. The endoscope of claim 7, the bending control lever further comprising:
a tightening member used to attach the connecting member to the shaft, the tightening member having an outer diameter that decreases toward the base.

9. The endoscope of claim 7, wherein the first annular surface, second annular surface and third annular surface each of which has a surface roughness set equal to or higher than remaining parts of the bending control lever.

10. The endoscope of claim 7, wherein the elastic cover further comprising a watertight attached portion that is attached on a side of the base.

11. The endoscope of claim 10, wherein the cover portion includes a valley portion between the watertight attached portion and the third attached portion.

12. The endoscope of claim 11, wherein the cover portion includes an outer peripheral wall portion rises from the watertight attached portion.

13. The endoscope of claim 12, wherein the valley portion includes a descending portion extending in a descending direction from a top portion of the outer peripheral wall portion.

14. The endoscope of claim 11, wherein the valley portion includes a planer portion.

15. The endoscope of claim 11, wherein the cover portion includes an inner peripheral wall portion disposed contiguously on an inner peripheral side of the valley portion.

16. The endoscope of claim 7, wherein the finger rest includes a convex portion extending toward the elastic cover.

17. The endoscope of claim 7, wherein the second annular surface has a smaller diameter than the first annular surface, the third annular surface has a greater diameter than the second annular surface.

18. The endoscope of claim 7, wherein the second annular surface has a greater diameter than the first annular surface, the third annular surface has a smaller diameter than the second annular surface.
